(19) United States
(12) Patent Application Publication
(10) Pub. No.: US 2026/0146950 A1
(43) Pub. Date: May 28, 2026

(54) SYSTEM AND METHOD FOR QUANTUM BIOSENSING OF SIGNAL CASCADES

(71) Applicant: Battelle Memorial Institute, Columbus, OH (US)

(72) Inventors: David Carl GLASBRENNER, Hilliard, OH (US); Yaser Hamdy HELAL, Columbus, OH (US); Ryan J. PATTON, Columbus, OH (US); Ryan J. RODRIGUEZ, Columbus, OH (US); Philip P. CHENEY, Bexley, OH (US)

(21) Appl. No.: 19/398,356

(22) Filed: Nov. 24, 2025

Related U.S. Application Data
(60) Provisional application No. 63/725,679, filed on Nov. 27, 2024.

(51) Int. Cl. G01N 21/64; G01N 33/50; G01N 33/58

(52) U.S. Cl. CPC G01N 21/6428; G01N 33/5091; G01N 33/582; G01N 2021/6439

(57) ABSTRACT

In an approach to quantum biosensing of signal cascades. The system includes a pump laser light source; a nonlinear optical crystal module; and an optical timing control; the system configured to: generate a two photon light; split the two photon light into a first photon light and a second photon light; generate a correlated single photon light by fixing a first timing of the first photon light and varying a second timing of the second photon light to achieve coincidence between the first photon light and the second photon light; excite a biomarker by the correlated single photon light to cause the biomarker to fluoresce; detect a biomarker fluorescence; and reconstruct a lifetime decay of a sample.

### CROSS-REFERENCE TO RELATED APPLICATIONS

The present application claims the benefit of the filing date of U.S. Provisional Application Ser. No. 63/725,679, filed Nov. 27, 2024, the entire teachings of which application is hereby incorporated herein by reference.

### TECHNICAL FIELD

The present disclosure relates generally to instrumentation and, more particularly, to a system and method for quantum biosensing of signal cascades.

### BACKGROUND

Current approaches for bio-sensing and imaging are limited in fields including DNA sequencing and drug delivery research. For DNA sequencing, the state-of-the-art requires the nucleic acid be removed from the organism(s) (e.g., eukaryote, bacteria, virus, fungi) being assessed and subsequently undergo an extensive series of laborious steps to prepare the sample for sequencing. This multi-step process incurs increased cost and time going from sample to answer, often taking an hour or more depending on the complexity of a given sample. Bioimaging has limited sensitivity to monitor drug metabolism and delivery at the molecular level to effectively understand drug pathway cascades.

### BRIEF DESCRIPTION OF THE DRAWINGS

Reference should be made to the following detailed description which should be read in conjunction with the following figures, wherein like numerals represent like parts.

FIG. 1 is a functional block diagram illustrating an example light source for a system for quantum biosensing of signal cascades, consistent with the present disclosure.

FIG. 2 is a functional block diagram illustrating an example system for quantum biosensing of signal cascades implemented on a microscope, consistent with the present disclosure.

FIG. 3 is an example of drug delivery pathways resolved by Time Correlated Single Photon Counting (TCSPC), consistent with the present disclosure.

FIG. 4 is an example flow of the final measurement in the method for quantum biosensing of signal cascades of FIG. 3, consistent with the present disclosure.

FIG. 5 is an example of applying hyperspectral imaging in the method for quantum biosensing of signal cascades of FIG. 3, consistent with the present disclosure.

FIG. 6 is a flowchart diagram depicting operations for the method for quantum biosensing of signal cascades, consistent with the present disclosure.

### DETAILED DESCRIPTION

The present disclosure is not limited in its application to the details of construction and the arrangement of components set forth in the following description or illustrated in the drawings. The examples described herein may be capable of other embodiments and of being practiced or being carried out in various ways. Also, it may be appreciated that the phraseology and terminology used herein is for the purpose of description and should not be regarded as limiting as such may be understood by one of skill in the art. Throughout the present disclosure, like reference characters may indicate like structure throughout the several views, and such structure need not be separately discussed. Furthermore, any particular feature(s) of a particular exemplary embodiment may be equally applied to any other exemplary embodiment(s) of this disclosure as suitable. In other words, features between the various exemplary embodiments described herein are interchangeable, and not exclusive.

Quantum effects, such as spin-spin coupling, magnetic resonance, and photonic coupling have the potential for increasing resolution and specificity to provide the capability to differentiate individual nucleotides for DNA sequencing and provide sensitivity to monitor drug pathways.

These quantum effects may be used in applications such as drug activity studies to detect cascading events. For example, in the process of drug interaction, they may provide better clarity, as current detection systems are too slow to detect these effects. In another example, current methods of DNA sequencing must remove the DNA from the organism. The disclosed techniques may allow for sequencing without removing the DNA from the organism.

Disclosed herein is a system and method for quantum biosensing of signal cascades including a quantum optical light source for time correlated photon microscopy and imaging, a technique that may increase the efficiency and resolution of two photon absorption at low photon energies. In an embodiment, the system and method disclosed herein is a quantum sensor utilizing detection of photon pairs in biologic systems to detect cascading events at a molecular level following small molecule perturbations (e.g., a drug or a toxin). In another embodiment, the system and method disclosed herein may be configured for drug delivery research, for example, to study an in vivo drug pathway. In yet another embodiment, the system may be configured for semiconductor metrology, for example, for integrated circuit failure analysis.

This disclosure describes novel instrumentation for an optical quantum sensor of a single photon and/or a photon pair. For this system, a source of photon pairs is disclosed. This may be achieved using a nonlinear crystal, which can generate pairs of photons that are time correlated with each other. The photons may be generated using a laser that is tuned to the appropriate wavelength, and the nonlinear crystal can be placed in a temperature-controlled environment to optimize the efficiency of the process. Also disclosed is a specialized optical setup to detect the photon pairs. This may involve using a set of lenses, mirrors, fiber optic components, polarization splitters, and/or bandwidth splitting components to direct the photons onto a detector (such as a photomultiplier tube or an avalanche photodiode). Of note, the detector needs to be placed in a low-noise environment, such as a cooled and/or vacuum chamber, to minimize external interference. To detect photon pairs in a biologic system, target molecules are labeled, such as known enzymes in a drug pathway cascade, in a mammalian cell line (e.g., HeLa) with fluorescent dyes (e.g., green fluorescent protein (GFP)), which can be excited by the photons. The photons that are emitted by the fluorescent markers may then be detected using the specialized optical setup described below. The data collected by the detector may then be used to determine whether the photons have been detected. This typically involves using statistical analysis techniques to look for correlations between the photons detected at different times and locations.

Existing methods of biosensing have limitations in sensitivity and resolution, limiting understanding and developments of single-cell and molecular scale interactions in biological processes. Quantum sensors have the potential to outperform classical sensors, and demonstrations of the quantum advantage in practical sensing applications are currently limited and emerging. As classical sensors are readily impacted by external noise and interference, use of quantum sensors that rely on the quantum states of individual particles are better suited to measure biologic processes. For example, quantum sensors may be used to measure the activity of enzymes and other proteins involved in drug metabolism and drug delivery. This can provide insights into how drugs are being processed by the body, and ultimately to design more effective drugs that are better tailored to individual patients. As the field of personalized medicine takes center light as the driving standard of care, quantum sensors will play an integral role in therapeutic implementation for an individual.

Disclosed herein are systems and methods for optical quantum sensing which may be applied to novel biosensing and imaging. In an embodiment, nucleotides in a DNA sequence are differentiated by leveraging the principles of quantum mechanics and the capabilities of Optically Detected Magnetic Resonance (ODMR). This approach exploits the unique quantum mechanical properties of each nucleotide/DNA molecule (e.g., chemical shift, spin-spin coupling, relaxation times, nuclear Overhauser effect, or quadrupolar interaction), providing a novel method for DNA sequencing.

In addition, disclosed is a quantum optical light source, which generates time correlated photo pairs. This light source may be used for two-photon absorption and achieve time correlated photon microscopy in a fluorescence-tagged sample for biosensing. The time correlated photon microscopy technique demonstrates increased efficiency of two-photon absorption at low incidence power, which may be applied to monitoring drug delivery at high resolution with reduced impacts due to phototoxicity.

Existing methods for DNA sequencing, such as next-generation sequencing (NGS), rely on biochemical reactions to determine the sequence of bases in a DNA molecule. Though these methods are highly effective and have revolutionized the field of genomics, they also have limitations. Most notably, NGS requires amplification of the original sequence in order to reach a limit of detection threshold towards determining the nucleotide information.

Similarly, existing methods for bioimaging have limitations in monitoring single-cell and molecular scale interactions to effectively understand interactions in drug delivery. Two-photon absorption is a ‘classical’ light source technique which provides high contrast and high resolution imaging, but is very inefficient, requiring high power laser light sources. A time correlated photon source significantly increases the probability of two-photon incidence at the sample, allowing for higher efficiency absorption at much lower laser powers, which is favorable for sensitive bio-samples.

Quantum imaging methods may include Time Correlated Single Photon Counting (TCSPC) and Quantum Imaging with photon pairs. One method of TCSPC is fluorescence imaging, which may include laser scanning TCSPC and two dimensional imaging using an array or a fiber bundle.

Laser scanning TCSPC is a technique used in laser-scanning microscopy to measure fluorescence decays and reconstruct the lifetime decay of a sample by using the timing of a pulsed excitation source with the timing of the arrival of single photons on a detector to reconstruct the lifetime decay over many events. Two dimensional imaging can be accomplished using a pixel array or a fiber bundle, but this is an expensive, innovative technology. Currently only a few suppliers make superconducting nanowire single-photon detector (SNSPD) pixel arrays, which are optical and near-infrared detectors that can detect single photons. Single-photon avalanche diode (SPAD) arrays are less efficient but are further along in maturation to pixel arrays. An intensified CMOS/CCD is less efficient and slower than the above but are commercially available.

Quantum imaging with photon pairs may include time correlated photon microscopy. Time correlated photon microscopy uses two-photon excitation of a fluorescence marker. Using a time correlated two-photon source may increase Signal to Noise Ratio (SNR) by the improved probability of two-photon interaction. In an embodiment, the disclosed system applies two-photon excitation to drug delivery research.

In drug delivery research, the two-photon system allows for real-time, high-resolution tracking of fluorescent biomarkers as they traverse cellular and tissue environments. The use of a superconducting nanowire single-photon detector array provides both spatial and temporal resolution, enabling the mapping of drug pathways (i.e., molecular events like enzymatic activation, receptor binding, neurotransmitter release, etc.) through monitoring of molecular interactions at the single-cell level. The system's ability to reconstruct the lifetime decay of fluorescence emissions further enhances the understanding of drug metabolism and transport mechanisms. This application is novel because it extends quantum biosensing to the direct observation and analysis of drug delivery pathways in vivo which could aid in better understanding off-target effects, toxicity, and mechanisms of action, leading to faster R&D downselection during pre-clinical trial research.

Hybrid time correlated imaging may include time correlation using photon pairs. When using a photon pair source to image a fluorescent tagged sample, the entanglement is broken by generation of the fluorescent photon, but time correlation is still possible. Using an illumination and detection protocol to correlate detections using time-gating for interaction versus non-interaction may reduce noise to the sub-shot noise limit for detection. Therefore, the repetition rate of the source needs to be longer than the fluorescence lifetime.

A second quantum imaging method is two dimensional TCSPC imaging, which may utilize an SNSPD pixel array, such as a multi-channel SNSPD using 6×6 nanowires organized in a square array, with each pixel corresponding to an SNSPD channel. A representative multi-channel SNSPD is produced by Single Quantum of The Netherlands. An SNSPD array allows spatial and temporal tracking of in vivo biomarkers on time scales comparable to the resolution limit of the SNSPD.

The disclosed system and method may utilize two photon absorption. In the two photon absorption, a fluorophore is excited by a specific wavelength, for example, one that responds to 785 nm. Direct 785 nm irradiation would be very efficient, however there may be circumstances where this wavelength is not optimal. Generating a two photon light with two photon excitation uses two 785 nm lights to yield a 1570 nm light to excite the fluorescence. Although this is inefficient, requiring more laser power, it does have several benefits, including that using a longer wavelength can be better for the sample when damage is a concern, better background light rejection, and contrast can be excellent with a two-photon Laser Scanning Microscopy (LSM). Using a biphoton source improves the probability of two-photon excitation. The “quantum entanglement” of the source only matters as much as the coincident timing of the photons. Considerations of illumination area and the timing of twin beams impacts the absorption rate and enhancement over conventional two-photon absorption.

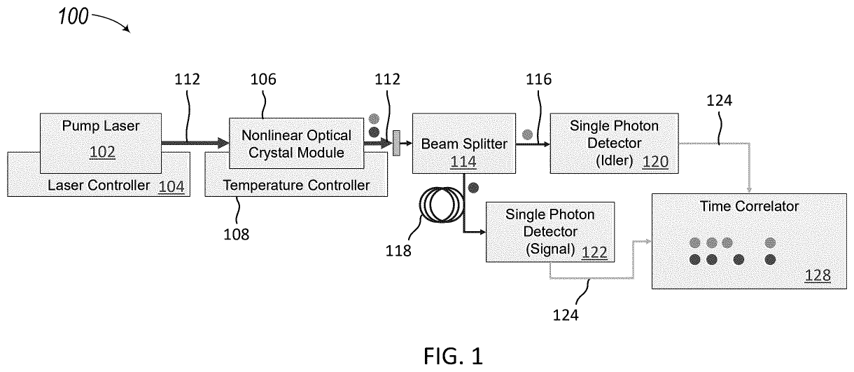

FIG. 1 is a functional block diagram illustrating an example light source 100 for a system for quantum biosensing of signal cascades, consistent with the present disclosure. The illustrative example light source 100 of FIG. 1 includes a pump laser 102 as the light source and a laser controller 104 to control the pump laser 102. The pump laser 102 is optically coupled to a nonlinear optical crystal module 106 by a polarization-maintaining optical fiber 112. In an embodiment, the nonlinear optical crystal module 106 may include any nonlinear crystal suitable for use in Spontaneous Parametric Down Conversion, for example, Periodically Poled Lithium Niobate (PPLN) or Beta Barium Borate (BBO). In other embodiments, however, any other suitable nonlinear crystals may be used. Polarization-maintaining optical fiber (PMF or PM fiber) is a single-mode optical fiber in which linearly polarized light, if properly launched into the fiber, maintains a linear polarization during propagation, exiting the fiber in a specific linear polarization state. The nonlinear optical crystal module 106 is used to create a pair of polarization correlated photon-pairs whose spectrum may be tuned by adjusting the temperature of the module. A temperature controller 108 is used to tune the nonlinear optical crystal module 106 to the desired spectrum.

The nonlinear optical crystal module 106 is coupled to a beam splitter 114 which splits the pair of polarization correlated photon-pairs into two separate paths. The beam splitter 114 directs one of the photons to an idler single photon detector 120 over a single-mode (SM) optical fiber cable 116. The beam splitter 114 directs the other photon to a signal single photon detector 122 over a variable length SM optical fiber cable 118. The idler single photon detector 120 and the signal single photon detector 122 are coupled to a time correlator 128 via SMA cables 124. The length of the variable length SM optical fiber cable 118 may be adjusted to achieve the desired delay to correlate the two photons. A time correlator 128 may be used to characterize the two photon sources to identify coincidence pairs of photons with respect to time and measure the correlation.

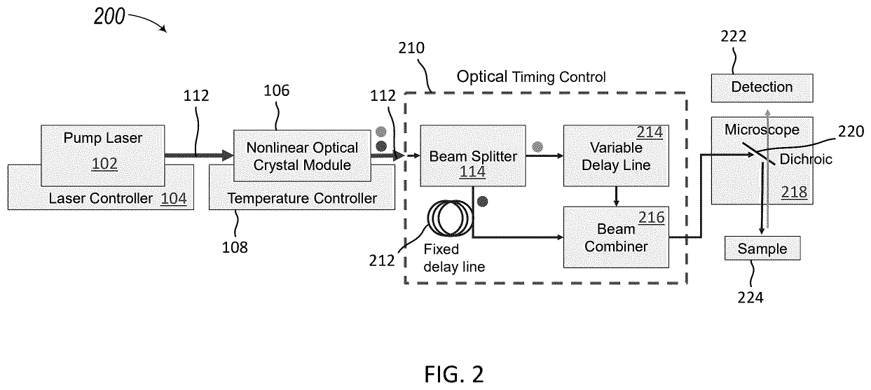

FIG. 2 is a functional block diagram illustrating an example system 200 for quantum biosensing of signal cascades implemented on a microscope, consistent with the present disclosure. The illustrative example system 200 of FIG. 2 includes the pump laser 102, the laser controller 104, the nonlinear optical crystal module 106, the PM optical fiber 112, and the temperature controller 108 from FIG. 1. The system 200, however, includes an optical timing control 210 to control the precise coincident timing to achieve efficient two-photon absorption.

The timing of the two beams is critical. To achieve the necessary coincident timing, one of the photon beams is fixed while the other is variable to ensure they arrive at the target at the same time. In illustrative example of FIG. 2, one photon beam is fixed in length, and therefore fixed delay, while the other photon beam has a variable transmission timing. The optical timing control 210 adjusts the variable transmission timing of the second beam to achieve coincidence.

At the output of the nonlinear optical crystal module 106 there may be a delay between orthogonally polarized photons in the PM optical fiber 112. To recombine the coincident timing of the photons, the delay of the first of the orthogonally polarized photons is exaggerated in one single mode fiber line, i.e., fixed delay line 212, and a variable delay line 214 is used to adjust the delay of the second of the orthogonally polarized photons. In an embodiment, the variable delay line 214 may be an electronic delay. In another embodiment, the variable delay line 214 may be a moving mirror to adjust the delay. In yet another embodiment, any appropriate variable delay may be used as would be known to one skilled in the art.

The light source output of a beam combiner 216 is coupled to a microscope 218 for fluorescence scanning imaging. The output of the light source may pass through a dichroic 220 to a sample 224. The detection 222 may be achieved either using a confocal setup or an imaging setup with either a camera or a point detector with good detection efficiency, ideally single photon detection capability.

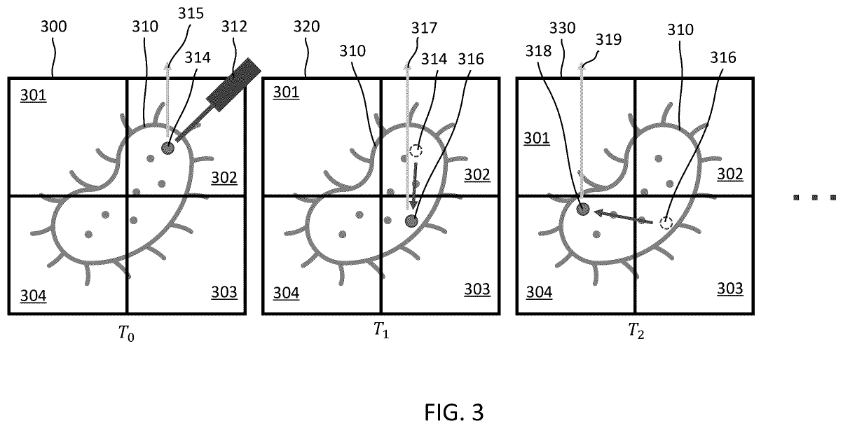

FIG. 3 is an example of drug delivery pathways resolved by TCSPC, consistent with the present disclosure. In an embodiment, the disclosed system continually fluoresces biomarkers injected into a cell 310 while imaging the cell 310 onto an SNSPD array. The array in this example is a 4×4 array consisting of quadrant-1 301, quadrant-2 302, quadrant-3 303, and quadrant- 4 304. The emission is collected by the SNSPD corresponding to the pixel channel. In the example of FIG. 3, at time T₀ 300 a fluorescent biomarker 312 is injected into a cell 310 at a first location 314.

In an embodiment, the fluorescent biomarker 312 may be, for example, a quantum dot (QD), a semiconductor nanocrystal used as a fluorescent probe for biological imaging. In other embodiments, the fluorescent biomarker 312 may be inorganic near-infrared II (NIR-II) fluorophores, for example, rare earth doped nanoparticles (RENPs), and/or single-walled carbon nanotubes (SWCNTs).

The first emission 315 is collected by the SNSPD corresponding to the pixel channel. At time T₁ 320, the fluorescent biomarker has progressed to a second location 316 with a second emission 317, and at time T₂ 330, the fluorescent biomarker has progressed to a third location 318 with a third emission 319.

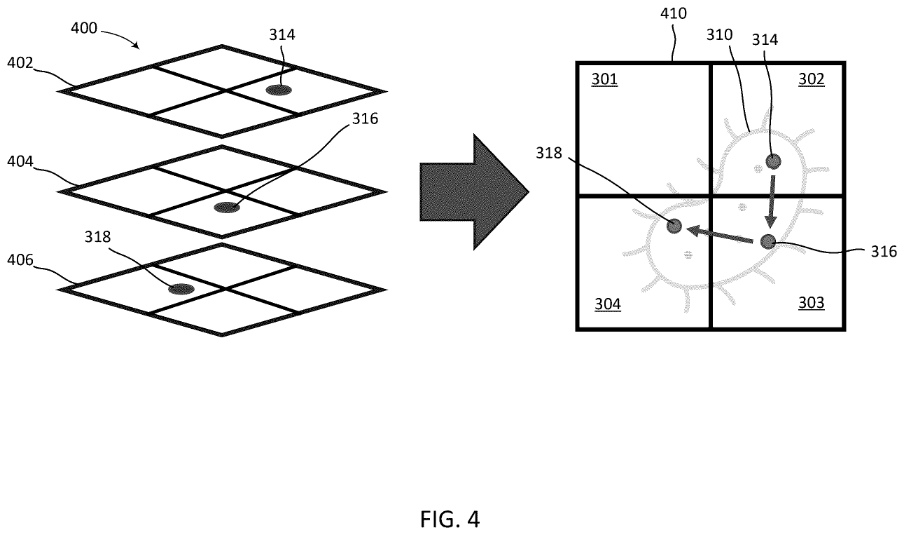

FIG. 4 is an example flow 400 of the final measurement in the method for quantum biosensing of signal cascades of FIG. 3, consistent with the present disclosure. In an embodiment, the movement of the biomarker 312 is tracked as the emission is collected. The final measurement is a data stack of individual 4×4 slices that combine to create a “movie” of the in vivo drug pathway. The first slice 402 shows the first location 314 of the biomarker 312 at time T₀ 300, the second slice 404 shows the second location 316 of the biomarker 312 at time T₁ 320, and the third slice 406 shows the third location 316 of the biomarker 312 at time T₂ 330. The corresponding movement of the biomarker 312 is illustrated in image 410 as the biomarker 312 progresses from the first location 314 at time T₀ 300 to the second location 316 at time T₁ 320, and the third location 316 of the biomarker 312 at time T₂ 330.

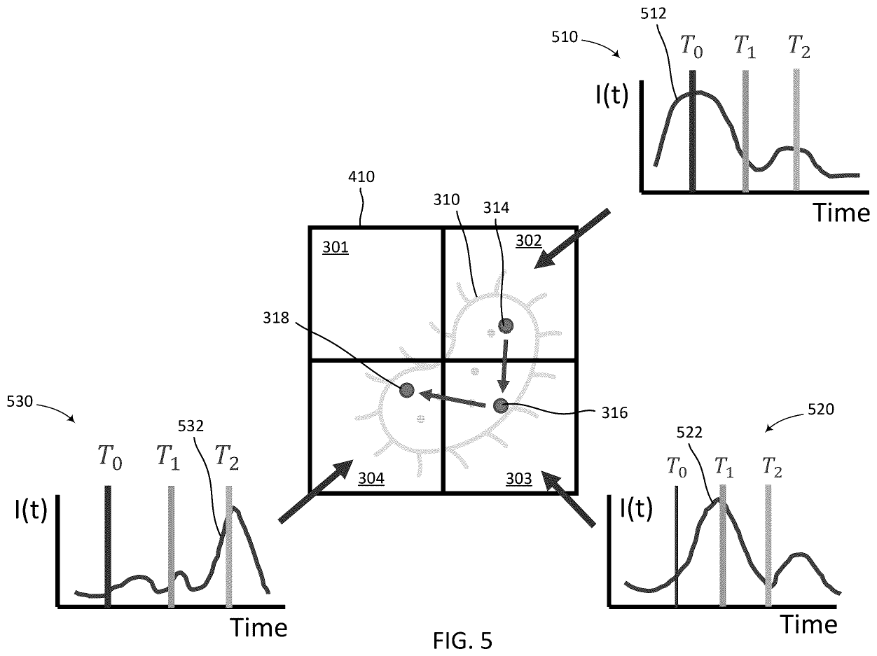

FIG. 5 is an example of applying hyperspectral imaging in the method for quantum biosensing of signal cascades of FIG. 4, consistent with the present disclosure. The example of FIG. 5 may illustrate, for example, a drug pathway through a cell 310. In the example of FIG. 5, the image 410 from FIG. 4 illustrates the movement of the biomarker 312 from the first location 314 in quadrant-2 302 through the second location 316 in quadrant-3 303 and ultimately arriving at the third location 318 in quadrant-4 304. A first chart 510, a second chart 520, and a third chart 530 are graphs of the intensity of fluorescence over time, where the vertical axis is the intensity of fluorescence and the horizontal axis is time.

The first chart 510 illustrates that at time T₀, the captured emissions show a peak in quadrant-2 302, represented by the blue channel. The second chart 520 illustrates that at time T₁, the captured emissions show a peak in quadrant-3 303, represented by the green channel. Finally, the third chart 530 illustrates that at time T₂, the captured emissions show a peak in quadrant-4 304, represented by the yellow channel. This illustrates that over the three time intervals, T₀, T₁, and T₂, the target drug has moved along the path from the first location 314 through the second location 316 and ultimately arriving at the third location 318 in quadrant-4 304.

It should be noted that the color channels in this example are intended to distinguish between the spectrum channels for the graph and are not representative of the actual spectrum.

In an embodiment, assigning the TCSPC data to the gridded pixel array may be applied to convolutional neural networks (CNNs) for temporal classifications.

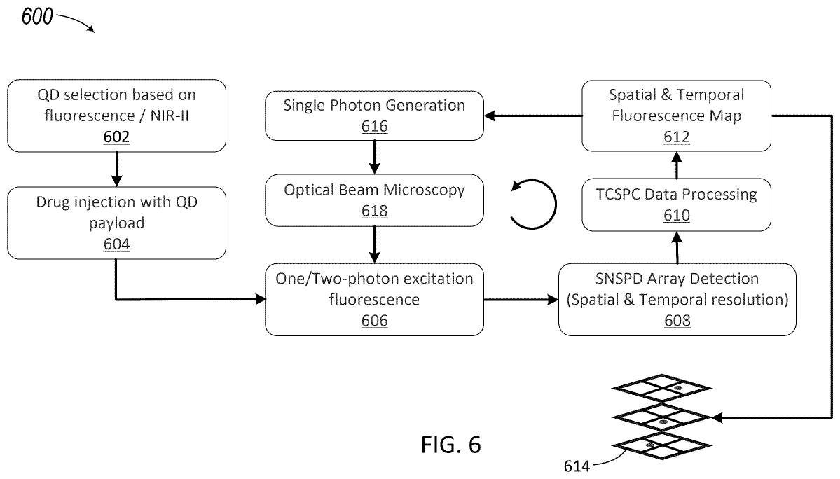

FIG. 6 is a flowchart diagram depicting the process 600 for one example embodiment of the method for quantum biosensing of signal cascades, consistent with the present disclosure. It should be appreciated that FIG. 6 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 600 includes selecting the biomarker (operation 602). In the illustrated example embodiment, a biomarker is selected based on the fluorescence. In an embodiment, the biomarker may be an inorganic NIR-II fluorophores which may include, but is not limited to, QDs, RENPs, and/or SWCNTs.

Process 600 also includes injecting the biomarker (operation 604). The biomarker payload selected in operation 602 is injected into the target, for example, a cell. In an embodiment, the biomarker may be selected from dyes and/or QDs with short emission lifetime to increase the timing resolution. Low quantum yields of NIR-II fluorophores limit penetration depth and cause low temporal resolution, using high QE SNSPD increases both the penetration depth and the temporal resolution. Therefore, in an embodiment, a high QE SNSPD may be selected. In addition, the QDs exhibit the highest fluorescence quantum yields (up to 15.5%).

Process 600 also includes one or two-photon excitation fluorescence (operation 606). The target is excited by a light source using either one photon or two photon excitation to cause the biomarker to fluoresce. Two photon microscopy may provide clearer images because they are prone to less photobleaching than single photon microscopy. Therefore, in an embodiment, two photon microscopy may be used.

Process 600 also includes SNSPD array detection (Spatial & Temporal resolution) (operation 608). The SNSPD array detects the fluorescence from the biomarker in the target. In an embodiment, the SNSPD array may provide both spatial and temporal resolution.

Process 600 also includes TCSPC data processing (operation 610). The TCSPC data processing may measure fluorescence decays and reconstruct the lifetime decay of a sample by using the timing of a pulsed excitation source with the timing of the arrival of single photons on a detector to reconstruct the lifetime decay over many events.

Process 600 also includes spatial & temporal fluorescence map (operation 612). The data processed by the TCSPC data processing in operation 610 is used to create a spatial and temporal fluorescence map of the movement of the biomarker within the target.

Process 600 also includes creating a multilayer data-stack of TCPSC images (operation 614). An example may be found in FIG. 4.

Process 600 also includes Single Photon Generation (Spatial & Temporal resolution) (operation 616). The optical timing control, e.g., the optical timing control 210 from FIG. 2, varies the timing of one of the photons to achieve coincidence between the two photons. The two photon beams are recombined, for example, by the beam combiner 216 of the optical timing control 210 from FIG. 2, into a single photon.

Process 600 also includes optical beam microscopy (operation 618). The process 600 may be applied to, for example, optical beam microscopy.

According to one aspect of the disclosure there is thus provided a system for quantum biosensing of signal cascades. The system includes a pump laser light source; a nonlinear optical crystal module; and an optical timing control; the system configured to: generate a two photon light; split the two photon light into a first photon light and a second photon light; generate a correlated single photon light by fixing a first timing of the first photon light and varying a second timing of the second photon light to achieve coincidence between the first photon light and the second photon light; excite a biomarker by the correlated single photon light to cause the biomarker to fluoresce; detect a biomarker fluorescence; and reconstruct a lifetime decay of a sample.

According to another aspect of the disclosure, there is thus provided a method of quantum biosensing of signal cascades. The method includes selecting a biomarker; injecting the biomarker into a target; generating a two photon light; splitting the two photon light into a first photon light and a second photon light; generating a correlated single photon light by fixing a first timing of the first photon light and varying a second timing of the second photon light to achieve coincidence between the first photon light and the second photon light; exciting the biomarker by the correlated single photon light to cause the biomarker to fluoresce; detecting a biomarker fluorescence; and reconstructing a lifetime decay of a sample.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

“Circuitry,” as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry and/or future computing circuitry including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DSP), field programmable gate array (FPGA), logic gates, registers, semiconductor device, chips, microchips, chip sets, etc.

The term “coupled” as used herein refers to any connection, coupling, link, or the like by which signals carried by one system element are imparted to the “coupled” element. Such “coupled” devices, or signals and devices, are not necessarily directly connected to one another and may be separated by intermediate components or devices that may manipulate or modify such signals.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles “a” and/or “an” and/or “the” to modify a noun may be understood to be used for convenience and to include one, or more than one, of the modified noun, unless otherwise specifically stated. The terms “comprising”, “including” and “having” are intended to be inclusive and mean that there may be additional elements other than the listed elements.

It will be appreciated by those skilled in the art that any block diagrams herein represent conceptual views of illustrative circuitry embodying the principles of the disclosure. Similarly, it will be appreciated that any block diagrams, flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

The flowchart and block diagrams in the Figures illustrate the architecture, functionality, and operation of possible implementations of systems and methods according to various embodiments of the present disclosure. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the disclosure. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A system for quantum biosensing of signal cascades, the system comprising:
a pump laser light source;
a nonlinear optical crystal module; and
an optical timing control;
the system configured to:
generate a two photon light;
split the two photon light into a first photon light and a second photon light;
generate a correlated single photon light by fixing a first timing of the first photon light and varying a second timing of the second photon light to achieve coincidence between the first photon light and the second photon light;
excite a biomarker by the correlated single photon light to cause the biomarker to fluoresce;
detect a biomarker fluorescence; and
reconstruct a lifetime decay of a sample.

2. The system of claim 1, wherein the nonlinear optical crystal module is selected from a group consisting of Periodically Poled Lithium Niobate (PPLN) and Beta Barium Borate (BBO).

3. The system of claim 1, further comprising:
a laser controller configured to control the pump laser light source.

4. The system of claim 1, further comprising:
a temperature controller configured to tune the nonlinear optical crystal module to a desired spectrum by adjusting a temperature of the nonlinear optical crystal module.

5. The system of claim 1, wherein the optical timing control further comprises:
a beam splitter;
a fixed delay line;
a variable delay line; and
a beam combiner.

6. The system of claim 5, wherein the variable delay line is an electronic delay.

7. The system of claim 5, wherein the variable delay line is a moving mirror.

8. The system of claim 1, wherein the biomarker is at least one of:
a quantum dot (QD), wherein the QD is a semiconductor nanocrystal used as a fluorescent probe for biological imaging;
inorganic near-infrared II (NIR-II) fluorophores;
rare earth doped nanoparticles (RENPs); and
single-walled carbon nanotubes (SWCNTs).

9. The system of claim 1, wherein reconstruct the lifetime decay of the sample further comprises:
reconstruct the lifetime decay of the sample by using a third timing of a pulsed excitation source with a fourth timing of an arrival of single photons on a detector to reconstruct the lifetime decay of the sample over many events.

10. The system of claim 1, wherein the biomarker fluorescence is detected by a superconducting nanowire single-photon detector (SNSPD) pixel array detector.

11. A method for quantum biosensing of signal cascades, the method comprising:
selecting a biomarker;
injecting the biomarker into a target;
generating a two photon light;
splitting the two photon light into a first photon light and a second photon light;
generating a correlated single photon light by fixing a first timing of the first photon light and varying a second timing of the second photon light to achieve coincidence between the first photon light and the second photon light;
exciting the biomarker by the correlated single photon light to cause the biomarker to fluoresce;
detecting a biomarker fluorescence; and
reconstructing a lifetime decay of a sample.

12. The method of claim 11, wherein the biomarker is at least one of:
a quantum dot (QD), wherein the QD is a semiconductor nanocrystal used as a fluorescent probe for biological imaging;
inorganic near-infrared II (NIR-II) fluorophores;
rare earth doped nanoparticles (RENPs); and
single-walled carbon nanotubes (SWCNTs).

13. The method of claim 11, wherein reconstructing the lifetime decay of the sample further comprises:
reconstructing the lifetime decay of the sample by using a third timing of a pulsed excitation source with a fourth timing of an arrival of single photons on a detector to reconstruct the lifetime decay of the sample over many events.

14. The method of claim 13, wherein the lifetime decay of the sample is reconstructed using a Time Correlated Single Photon Counting (TCSPC).

15. The method of claim 14 further comprising:
creating a multilayer data-stack of TCPSC images.

16. The method of claim 14, wherein the biomarker fluorescence is detected by a superconducting nanowire single-photon detector (SNSPD) pixel array detector.

17. The method of claim 16, wherein the TCSPC uses a first excitation timing of the pulsed excitation source with a second excitation timing of the arrival of the single photons on the SNSPD detector.

18. The method of claim 17, further comprising:
using TCSPC data processing to create a spatial and temporal fluorescence map of a movement of the biomarker within the target.

19. The method of claim 11, wherein generating the two photon light further comprises:
using two 785 nm lights to yield a 1570 nm light to excite the biomarker fluorescence of the biomarker.

20. The method of claim 11, further comprising:
tracking of the biomarker using optical beam microscopy as the biomarker traverses cellular and tissue environments for drug delivery research.
